(19) United States
(12) Patent Application Publication
(10) Pub. No.: US 2025/0375238 A1
(43) Pub. Date: Dec. 11, 2025

(54) GUIDEWIRES AND METHODS OF USING SAME

(71) Applicant: BARD PERIPHERAL VASCULAR, INC., Franklin Lakes, NJ (US)

(72) Inventors: Breanna Simpson, Austin, TX (US); Peter Oladipo Akerele-Ale, Phoenix, AZ (US); Andy Moll, Mesa, AZ (US); Alex Palmer, Scottsdale, AZ (US); Olivia R. Palmer, Baltimore, MD (US); Kristin N. Rominger, Bloomingdale, NJ (US)

(73) Assignee: BARD PERIPHERAL VASCULAR, INC., Franklin Lakes, NJ (US)

(21) Appl. No.: 18/877,399

(22) PCT Filed: Jul. 15, 2022
(86) PCT No.: PCT/US22/37298

§ 371 Date: Dec. 20, 2024

(51) Int. Cl. A61B 18/14; A61B 18/00; A61F 2/95; A61M 25/09

(52) U.S. Cl. CPC A61B 18/14; A61F 2/95; A61M 25/09; A61B 2018/00345; A61B 2018/00404; A61B 2018/144; A61M 2025/09133; A61M 2025/09175; A61M 2210/12

(57) ABSTRACT

A guidewire includes a body and a tip. The tip is configured to be energized by an RF generator connectable to the body. The guidewire is configured to tunnel through an internal lumen of a blood vessel, tunnel through a wall of the blood vessel, and tunnel through non-vascular tissue outside of the blood vessel.

### TECHNICAL FIELD

The present disclosure relates to assemblies, systems, and methods for tunneling through non-vascular tissue, and more particularly assemblies, systems, and methods with guidewires for tunneling through non-vascular tissue.

### BACKGROUND

Guidewires are generally advanced into a vasculature of a patient and directed to a desired site to perform a medical treatment, such as, but not limited to, formation of a fistula, balloon angioplasty, and deployment of a stent or graft. After advancement of a guidewire to a desired location, an additional device, such as a catheter may be advanced over the guidewire to the site for medical treatment. The catheter may include or carry the one or more instruments, such as an electrode, deployable balloon, stent, graft, and/or the like to the site for medical treatment. Guidewires, therefore, function as track through a patient's vasculature that catheters and other instruments or devices, which may be less maneuverable on their own, may be advanced over. Guidewire advancement and tracking may allow users to conduct minimally invasive surgeries and procedures. It may therefore be useful to find guidewire designs and methods that allow users access to portions of a patient's anatomy that they would otherwise not have access to.

### SUMMARY

One challenging aspect in medical treatments involving guidewires is being limited in the type or amount of tissue that a guidewire can be advanced through, therefore also limiting a user's ability to place additional instruments over the guidewire into the non-traversable tissue. Accordingly, a need exists for alternative systems, methods, and guidewires for granting users access to and through tissue previously difficult to reach and/or penetrate. Embodiments of the present disclosure are directed to improvements over the above limitations by providing guidewires configured for tunneling through, for example, non-vascular tissue.

In one embodiment, a method of bypassing an occlusion includes inserting a guidewire into a vessel, directing the guidewire into tissue surrounding the vessel at a first point, and tunneling through the tissue surrounding the vessel with the guidewire. The method also includes directing the guidewire into the vessel at a second point to form a passageway through the tissue surrounding the vessel from the first point to the second point. The guidewire is RF energized.

In another embodiment, a method of bridging a gap between a first vessel and a second vessel includes inserting a guidewire into the first vessel, directing the guidewire out of the first vessel at a first point, and tunneling through tissue separating the first vessel and the second vessel with the guidewire. The method also includes directing the guidewire into the second vessel at a second point to form a passageway from the first vessel to the second vessel through the tissue separating the first vessel and the second vessel. The guidewire is RF energized.

In another embodiment, a guidewire includes a body and a tip. The tip is configured to be energized by an RF generator connectable to the body. The guidewire is configured to tunnel through an internal lumen of a blood vessel, tunnel through a wall of the blood vessel, and tunnel through non-vascular tissue outside of the blood vessel.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a side view of a guidewire, according to one or more embodiments shown and described herein;

FIG. 2 schematically depicts an axial cross section of the guidewire of FIG. 1 taken along line A-A of FIG. 1, according to one or more embodiments shown and described herein;

FIG. 3 schematically depicts an axial cross section of the guidewire of FIG. 1 taken along line A-A of FIG. 1, according to one or more embodiments shown and described herein;

FIG. 4 schematically depicts an axial cross section of the guidewire of FIG. 1 taken along line A-A of FIG. 1, according to one or more embodiments shown and described herein;

FIG. 5 schematically depicts an axial cross section of the guidewire of FIG. 1 taken along line A-A of FIG. 1, according to one or more embodiments shown and described herein;

FIG. 6 schematically depicts an axial cross section of the guidewire of FIG. 1 taken along line A-A of FIG. 1, according to one or more embodiments shown and described herein;

FIG. 7 schematically depicts a side view of a guidewire, according to one or more embodiments shown and described herein;

FIG. 8 schematically depicts a cross section of the guidewire of FIG. 7 taken along line 8-8 of the guidewire of FIG. 7, according to one or more embodiments shown and described herein;

FIG. 9 schematically depicts a side view of a guidewire, according to one or more embodiments shown and described herein;

FIG. 10 schematically depicts a side view of a guidewire, according to one or more embodiments shown and described herein;

FIG. 11 schematically depicts a passageway formed by a guidewire through non-vascular tissue between two points of a vessel, according to one or more embodiments shown and described herein;

FIG. 12 schematically depicts a catheter, including an extension device and a stent graft, positioned in the passageway of FIG. 11, according to one or more embodiments shown and described herein;

FIG. 13 schematically depicts a stent graft deployed in the passageway of FIG. 11, according to one or more embodiments shown and described herein;

FIG. 14 schematically depicts a passageway through non-vascular tissue formed by a guidewire between a first point of a first vessel and a second point of a second vessel, according to one or more embodiments shown and described herein;

FIG. 15 schematically depicts a stent graft deployed in the passageway of FIG. 14, according to one or more embodiments shown and described herein; and

FIG. 16 schematically depicts a first catheter and a second catheter positioned in the first vessel and the second vessel of FIG. 14, according to one or more embodiments shown and described herein

FIG. 17 schematically depicts the first catheter and the second catheter of FIG. 16 forming a fistula between the first vessel and the second vessel of FIG. 14 at the passageway of FIG. 14, according to one or more embodiments shown and described herein.

### DETAILED DESCRIPTION

Embodiments described herein are directed to tunneling guidewires and methods of using the same. In some embodiments, the guidewires and methods may be used to form a fistula between two blood vessels. In some embodiments, the guidewires and methods may be used to bypass an occlusion.

Guidewires may be used to form a track for one or more other medical devices to be passed over. However, guidewires may be limited in the amount and density of tissue they can tunnel through, thereby limiting the ability for the one or more other medical devices to track the guidewire into certain tissues. The embodiments described herein address the one or more aforementioned limitations. In particular, the guidewires described herein may include a body and an RF energized tip. The guidewires herein may be particularly configured to tunnel through an internal lumen of a blood vessel, tunnel through a wall of the blood vessel, and tunnel through non-vascular tissue outside of the blood vessel. Non-vascular tissue, as used herein, may generally relate to any tissue in the body that is not a vein, an artery, or a capillary. Examples of non-vascular tissue that the guidewires herein may tunnel through include connective tissue (e.g. fascias, adipose tissue), pedicle bundles, epithelial tissue, skeletal muscle, smooth muscle, cardiac muscle, and nerve tissue (e.g. brain tissue). The guidewires herein may tunnel through dense non-vascular tissue that is unable to be tunneled through with current guidewire designs. Moreover, the guidewires herein may tunnel through distances of non-vascular tissue that are also unable to be tunneled through with current guidewire designs. Using the guidewires herein to tunnel through the greater distances or densities of tissue may enable bypass procedures where a stent graft is passed from a first point of an occluded blood vessel, through a passageway in surrounding tissue formed by the guidewire, and to a second point of the occluded blood vessel to bypass the occlusion. Using the guidewires herein to tunnel through the greater distances or densities of tissue may enable fistula-forming procedures where a fistula is formed between a first vessel and a second vessel separated by 5 mm of tissue. To increase the user control of the guidewires herein, the guidewires may be steered by an external magnet and/or directional fibers running through at least part of the length of the guidewires. Various embodiments will now be described in greater detail below with reference to the figures.

As used herein, the term “proximal” means closer to or in the direction of an origin of an element, such as a guidewire. The origin of a guidewire may be a handle or other user-manipulated portion of the guidewire. The term “distal” means further from the origin, or handle, of the guidewire. Put another way, the term “distal” means closer to or in the direction of a tip of a guidewire, which is separated from a handle of the guidewire by the length of the guidewire body.

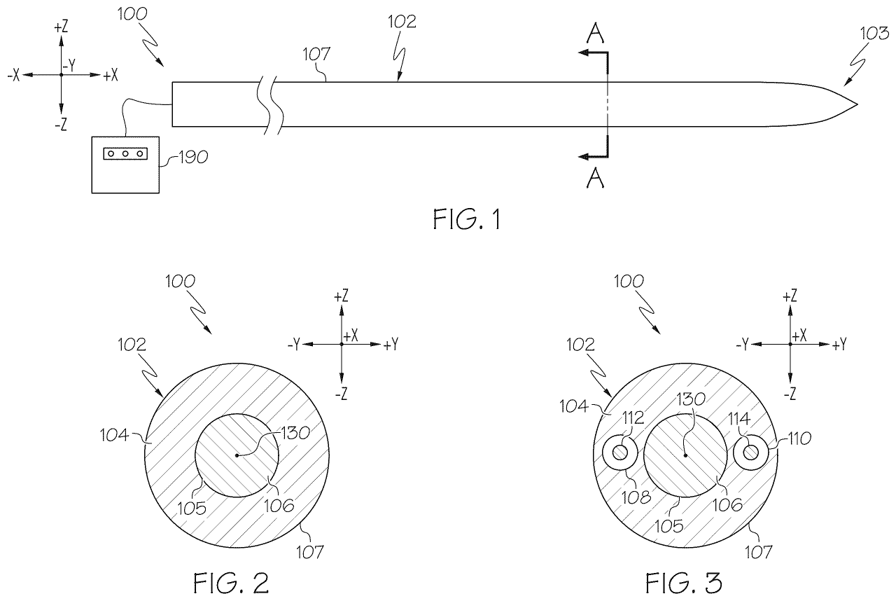

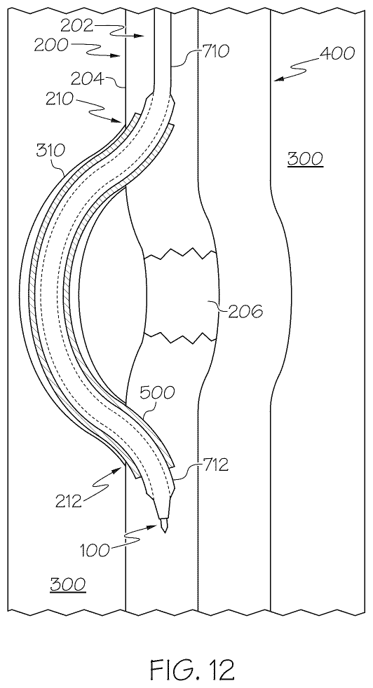

Referring now to FIG. 1 a guidewire 100 is depicted. The guidewire 100 includes a body 102 and a tip 103 positioned at a distal end of the body 102. The tip 103 may be pointed, rounded, or take any shape to assist in tunneling through tissue, as desired. The guidewire 100, and particularly the tip 103 of the guidewire 100, may be RF energized. That is, the guidewire 100, at its proximal end, may be coupled to an RF generator 190, and conduct RF energy to the tip 103 of the guidewire 100 (i.e. the guidewire 100 comprises or consists a conductive material so that RF energy may be conducted from the proximal end of the guidewire to the tip 103). That is, the RF generator 190 may be physically and/or electrically coupled to the body 102 of the guidewire 100 at a proximal end of the body 102, such as at or through a handle of the guidewire 100. The body 102 may then conduct RF energy to the tip 103. The body 102 of the guidewire 100 may be any desirable length. The body 102 of the guidewire 100 may be a length that allows the guidewire 100 to be inserted into a patient's body, and particularly into a patient's vasculature at a first point, and advanced from the first point, through the vasculature, to any desirable second point. In embodiments discussed herein, the body 102 of the guidewire 100 includes a predominantly circular cross section. However, it should be appreciated that the body 102 of the guidewire 100 may have any desirable cross-sectional shape. The circumference, or perimeter, of the guidewire 100 is generally small enough such that the guidewire 100 may be inserted and advanced through select portions of a patient's anatomy, including the vasculature. The circumference, or perimeter, of the guidewire 100 is also small enough to allow a catheter 710 (FIG. 12) or other medical device to be passed over the guidewire 100 in the same anatomy. That is, the catheter 710 (FIG. 12) may have a larger circumference, or perimeter, than the guidewire 100 such that the guidewire 100 may pass through an internal lumen of the catheter 710 (FIG. 12).

Referring now to FIGS. 1 and 2, a cross section of the guidewire 100, according to a first embodiment, is depicted about line A-A of FIG. 1. The guidewire 100 may include a core 106 and a shell 104. The shell 104 may include a core passageway 105 extending therethrough. The core passageway 105 may extend the entire length of the guidewire 100 (e.g. in the direction of the x-axis of the coordinate axes of FIG. 1). Therefore, in embodiments, the core passageway 105 may extend through the body 102 of the guidewire 100 and to a distal end of the distal tip 103 of the guidewire 100. In other embodiments, the core passageway 105 may extend through the body 102 of the guidewire 100 to a distal end of the body 102 of the guidewire 100. The core 106 may extend the entire length of the core passageway 105. The core 106 is positioned within the core passageway 105 of the shell 104. Therefore, the shell 104 may be positioned radially outward of the core 106. The shell 104 may define an exterior surface 107 of the guidewire 100. The core 106 and shell 104 may be concentric. In embodiments, the core 106 and the shell 104 may be concentric about a longitudinal centerline 130 of the guidewire 100. The core 106 and the shell 104 may be different materials. The core 106 may include a ferrous metal. For example, the core 106 may include any non-alloy steels and/or alloy steels. The core 106 may include low carbon steel, medium carbon steel, high carbon steel, chromium, manganese, nickel, silicon, titanium, vanadium, molybdenum, and/or any combinations thereof. As explained in greater detail herein, the core 106 may be made of any magnetic material that allows for magnetic steering or manipulation of the guidewire 100. The shell 104 may include a refractory metal. The shell 104 may include tungsten, rhenium, tantalum, molybdenum, niobium, and/or any combinations thereof. As explained in greater detail herein, the shell 104 may include any material with a sufficiently high melting point to conduct RF energy from the RF generator 190 to the tip 103 of the guidewire 100 without sustaining any structural deformations from the RF energy. The shell 104 allows the guidewire 100 to reach temperatures high enough to ablate tissue.

Referring now to FIGS. 1 and 3, another cross section of the guidewire 100, according to a second embodiment, is depicted about line A-A of FIG. 1. The guidewire 100 may include a first internal lumen 108 and a second internal lumen 110. The first internal lumen 108 and the second internal lumen 110 may extend from a proximal end of the guidewire 100 to a point within the body 102 of the guidewire 100. The first internal lumen 108 and the second internal lumen 110 may extend from a proximal end of the guidewire 100 to a distal end of the body 102 of the guidewire 100. The first internal lumen 108 and the second internal lumen 110 may extend from a proximal end of the guidewire 100 to or partially through, the tip 103 of the guidewire 100. The first internal lumen 108 and the second internal lumen 110 may be positioned diametrically opposite of each other. In other words, the first internal lumen 108 and the second internal lumen 110 may be positioned 180 degrees relative to each other. The guidewire 100 may include more or less than two internal lumens.

The first internal lumen 108 and the second internal lumen 110 may be formed in the shell 104. That is, the first internal lumen 108 and the second internal lumen 110 may be bounded by the material of the shell 104. The first internal lumen 108 and the second internal lumen 110 may be formed within the shell 104 of the guidewire 100 radially outward any distance from the longitudinal centerline 130 of the guidewire 100.

One or more directional fibers may be disposed within each internal lumen. The directional fibers may be ultra-high-molecular-weight polyethylene (UHMWPE) fiber, such as Dyneema® fiber, micro core wires, or any other material with a high pull strength. For instance, a first directional fiber 112 may be positioned within the first internal lumen 108, and a second directional fiber 114 may be positioned within the second internal lumen 110. The directional fibers may be fixed to a point within each of their respective internal lumens. In other words, the directional fibers may be fixed to points of the shell 104 that define each of their respective internal lumens.

With specific reference to the first directional fiber 112 within the first internal lumen 108, the first directional fiber 112 may be connected to an interior wall of the first internal lumen 108, or in other words, a surface of the shell 104 that defines the first internal lumen 108. The first directional fiber 112 may be connected to an interior wall of the first internal lumen 108 with a suitable polymer or adhesive, such as glue, heat shrunk plastic wrap, and the like. The first directional fiber 112 may be connected to an interior wall of the first internal lumen 108 at a distal end of the first internal lumen 108. In other words, the first directional fiber 112 may be fixed at a point within the first internal lumen 108 nearest to or within the tip 103 of the guidewire 100.

With specific reference to the second directional fiber 114 within the second internal lumen 110, the second directional fiber 114 may be connected to an interior wall of the second internal lumen 110, or in other words, a surface of the shell 104 that defines the second internal lumen 110. The second directional fiber 114 may be connected to an interior wall of the second internal lumen 110 with a suitable polymer or adhesive, such as glue, heat shrunk plastic wrap, and the like. The second directional fiber 114 may be connected to an interior wall of the second internal lumen 110 at a distal end of the second internal lumen 110. In other words, the second directional fiber 114 may be fixed at a point within the second internal lumen 110 nearest to or within the tip 103 of the guidewire 100.

The directional fibers may extend though the length of the guidewire 100 to the proximal end of the guidewire 100. More particularly, the directional fibers may extend proximally from the proximal end of the guidewire 100, allowing a user to selectively manipulate each directional fiber. With specific reference to the first directional fiber 112 within the first internal lumen 108, by pulling a proximal end of the first directional fiber 112 extending from the proximal end of the guidewire 100, a user may impart a force on the guidewire 100 at the point of connection between the first directional fiber 112 and the first internal lumen 108. Particularly, by pulling the first directional fiber 112, the user may steer or direct the guidewire 100, and specifically the tip 103, in the −y direction of the coordinate axes of FIG. 3. Through rotation of the guidewire 100 via a handle at a proximal end of the guidewire 100, it should be appreciated that the first internal lumen 108 and/or the second internal lumen 110 may be positioned at any relative angle in the y-z plane of the coordinate axes of FIG. 3 such that pulling of the directional fibers 112 and/or 114 may steer the guidewire 100 in any desirable direction.

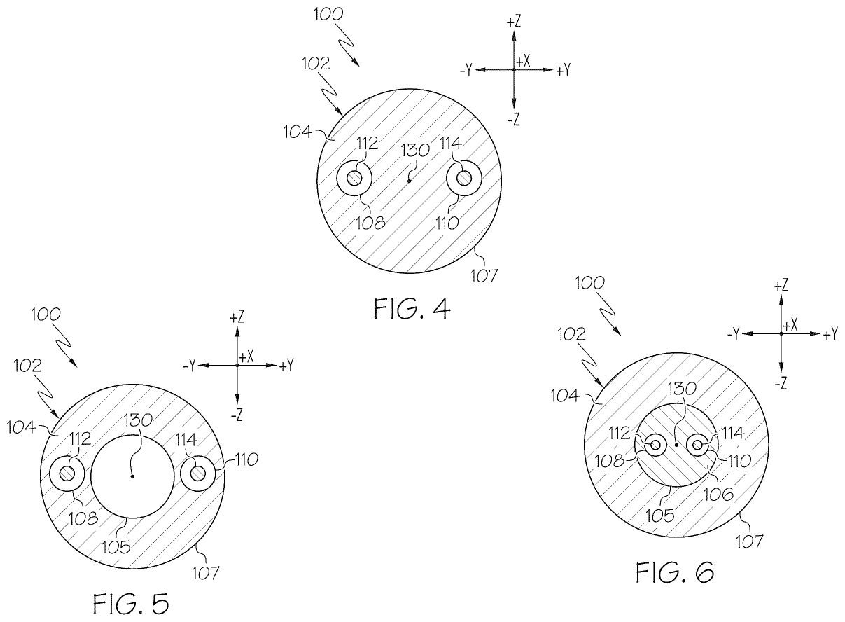

Referring now to FIG. 4, another cross section of the guidewire 100, according to a third embodiment, is depicted about line A-A of FIG. 1. Particularly, in embodiments, the guidewire 100 may not include the core 106 (FIGS. 2, 3). Instead, the guidewire 100 may only include the shell 104. In such embodiments, the shell 104 may define the first internal lumen 108 and/or the second internal lumen 110, as discussed with reference to FIG. 3. As described above with respect to FIG. 3, the first directional fiber 112 and the second directional fiber 114 may be positioned within the first internal lumen 108 and the second internal lumen 110, respectively. Moreover, as similarly discussed with reference to FIG. 3, the first directional fiber 112 and the second directional fiber 114 may be fixed to a point within each of their respective internal lumens.

Referring now to FIG. 5, another cross section of the guidewire 100, according to a fourth embodiment, is depicted about line A-A of FIG. 1. Particularly, in embodiments, the guidewire 100 may not include the core 106 (FIGS. 2, 3). Instead, the guidewire 100 may only include the shell 104. However, the guidewire 100 may still include the core passageway 105. The core passageway 105, may therefore, be void of any material. In such embodiments, the shell 104 may define the first internal lumen 108 and/or the second internal lumen 110, as discussed with reference to FIG. 3. As described above with respect to FIG. 3, the first directional fiber 112 and the second directional fiber 114 may be positioned within the first internal lumen 108 and the second internal lumen 110, respectively. Moreover, as similarly discussed with reference to FIG. 3, the first directional fiber 112 and the second directional fiber 114 may be fixed to a point within each of their respective internal lumens.

Referring now to FIG. 6, another cross section of the guidewire 100, according to a fifth embodiment, is depicted about line A-A of FIG. 1. The first internal lumen 108 and the second internal lumen 110 may be formed in the core 106. That is, the first internal lumen 108 and the second internal lumen 110 may be bounded by the material of the core 106. The first internal lumen 108 and the second internal lumen 110 may be formed within the core 106 of the guidewire 100 radially outward any distance from the longitudinal centerline 130 of the guidewire 100. As discussed above, the directional fibers may be fixed to a point within each of their respective internal lumens. In other words, the directional fibers may be fixed to points of the core 106 that define each of their respective internal lumens. With specific reference to the first directional fiber 112 within the first internal lumen 108, the first directional fiber 112 may be connected to an interior wall of the first internal lumen 108, or in other words, a surface of the core 106 that defines the first internal lumen 108.

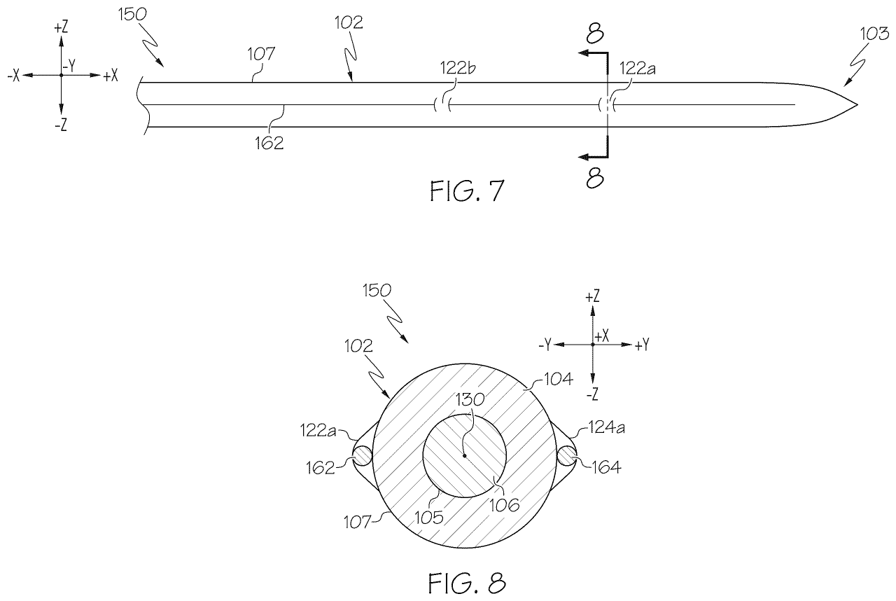

Referring now to FIG. 7, another embodiment of a guidewire 150 is schematically depicted. The guidewire 150 may generally resemble the guidewire 100 discussed with respect to FIGS. 1-6, except as noted herein. The guidewire 150 may include one or more directional fibers fixed to the exterior surface 107 of the guidewire 150. The directional fibers of the guidewire 150 resemble the directional fibers 112, 114 (FIGS. 3-6) discussed above. Therefore, the above description of the directional fibers 112, 114 (FIGS. 3-6) applies to the directional fibers of the guidewire 150 unless otherwise noted. More particularly, the guidewire 150 may include a first directional fiber 162. The first directional fiber 162 may extend proximally from a proximal end of the body 102 of the guidewire 150, allowing a user to manipulate the first directional fiber 162. The first directional fiber 162 may extend at least partially along the length of the guidewire 150. The first directional fiber 162 may extend along the length of the body 102 of the guidewire 150. The first directional fiber 162 may further extend at least partially along the length of the tip 103 of the guidewire 150.

The first directional fiber 162 may be coupled to the exterior surface 107 of the guidewire 150. The first directional fiber 162 may be coupled to the exterior surface 107 of the guidewire 150 with a suitable polymer or adhesive, such as glue, heat shrunk plastic wrap, and the like. The first directional fiber 162 may be coupled to the exterior surface 107 of the guidewire 150 at the distal end of the first directional fiber 162. The first directional fiber 162 may be coupled to the exterior surface 107 of the guidewire 150 at a point along the body 102 of the guidewire 150. The first directional fiber 162 may be coupled to the exterior surface 107 of the guidewire 150 at a distal end of the body 102 of the guidewire 150. The first directional fiber 162 may be coupled to the exterior surface 107 of the guidewire 150 at a point along the tip 103 of the guidewire 150.

The guidewire 150 may include one or more loops 122a, 122b along the exterior surface 107 of the guidewire 150 to maintain the first directional fiber 162 predominantly against the exterior surface 107 of the guidewire 150. The one or more loops 122a, 122b may be positioned along the body 102 and/or tip 103 of the guidewire 150. In embodiments, the one or more loops 122a, 122b may be made of the same material as the shell 104 of the guidewire 150. In embodiments, the one or more loops 122a, 122b may be metal, plastic, composite, and/or the like.

Referring now to FIG. 8, a cross section of the guidewire 150, according to embodiments, is depicted about line 8-8 of FIG. 7. In embodiments, and as depicted, the one or more loops may include a first loop 122a and a second loop 124a along and extending from the exterior surface 107 of the body 102 of the guidewire 150. The first loop 122a and the second loop 124a may be positioned diametrically opposite of each other. In other words, the first loop 122a and the second loop 124a may be positioned 180 degrees relative to each other. The first directional fiber 162 may be maintained against the exterior surface 107 of the guidewire 150 by the first loop 122a, and a second directional fiber 164 may be maintained against the exterior surface 107 of the guidewire 150 by the second loop 124a. It should be appreciated, however, that the guidewire 150 may include any number of directional fibers, and each directional fiber may be maintained against the exterior surface 107 of the guidewire 150 by one or more loops.

Referring to FIGS. 7 and 8, similar to the first directional fiber 162, the second directional fiber 164 may extend proximally from a proximal end of the body 102 of the guidewire 150, allowing a user to manipulate the second directional fiber 164. The second directional fiber 164 may extend at least partially along the length of the guidewire 150. The second directional fiber 164 may extend along the length of the body 102 of the guidewire 150. The second directional fiber 164 may further extend at least partially along the length of the tip 103 of the guidewire 150. The second directional fiber 164 may be coupled to the exterior surface 107 of the guidewire 150. The second directional fiber 164 may be coupled to the exterior surface 107 of the guidewire 150 with a suitable polymer or adhesive, such as glue, heat shrunk plastic wrap, and the like. The second directional fiber 164 may be coupled to the exterior surface 107 of the guidewire 150 at the distal end of the second directional fiber 164. The second directional fiber 164 may be coupled to the exterior surface 107 of the guidewire 150 at a point along the body 102 of the guidewire 150. The second directional fiber 164 may be coupled to the exterior surface 107 of the guidewire 150 at a distal end of the body 102 of the guidewire 150. The second directional fiber 164 may be coupled to the exterior surface 107 of the guidewire 150 at a point along the tip 103 of the guidewire 150.

As depicted in FIG. 8, the guidewire 150 may include the core passageway 105, with the core 106 positioned therein, and the shell 104, as discussed with respect to FIGS. 1-3. However, it should be appreciated that in embodiments, similar to those discussed with reference to FIG. 4, the guidewire 150 may not include the core 106 or the core passageway 105. Instead, the guidewire 150 may only include the shell 104. That is, the full cross-sectional area of the guidewire 150 may be the material of the shell 104. In yet other embodiments, similar to those discussed with reference to FIG. 5, the guidewire 150 may include the core passageway 105 within the shell 104. However, the guidewire 150 may not include the core 106, such that the core passageway 105 is void of material.

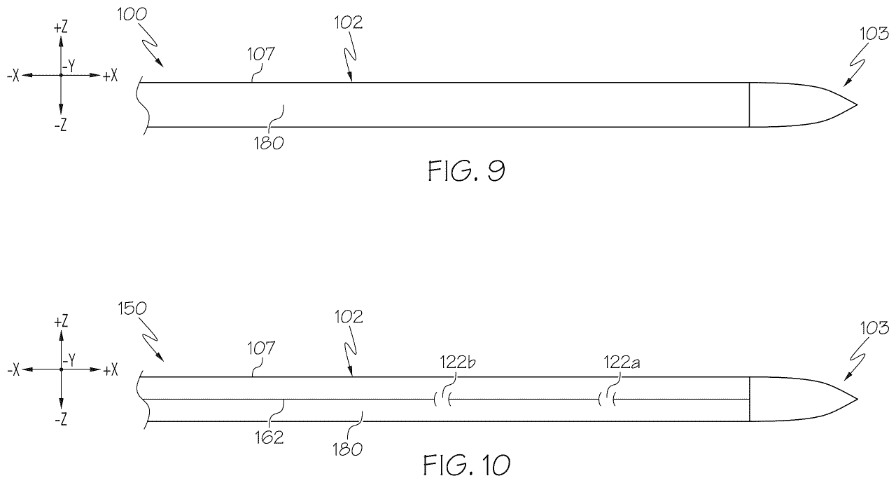

In any of the above-described embodiments, the body 102 of the guidewire 100, 150 may be coated in a heat and/or electrical insulating material 180. The insulating material 180 may include polymide, polytetrafluoroethylene, pebax, parylene, and/or perfluoroalkoxy. For instance, with reference to FIG. 9, the guidewire 100 of FIG. 1 is depicted with the insulating material 180 extending along and coating the body 102 of the guidewire 100. In such embodiments, the insulating material 180 may define the exterior surface 107 of the body 102 of the guidewire 100, but may not extend over the tip 103 of the guidewire 100. Similarly, with reference to FIG. 10, the guidewire 150 of FIG. 7 is depicted with the insulating material 180 extending along the body 102 of the guidewire 150. In such embodiments, the insulating material 180 may define the exterior surface 107 of the body 102 of the guidewire 100, but may not extend over the tip 103 of the guidewire 150. With the insulating material 180 not coating the tip 103 of the guidewire 100, 150, the tip 103 of the guidewire 100, 150 may be RF energized to enable the guidewire 100, 150 to tunnel through particularly dense tissue that the guidewire 100, 150 would otherwise be unable to tunnel through.

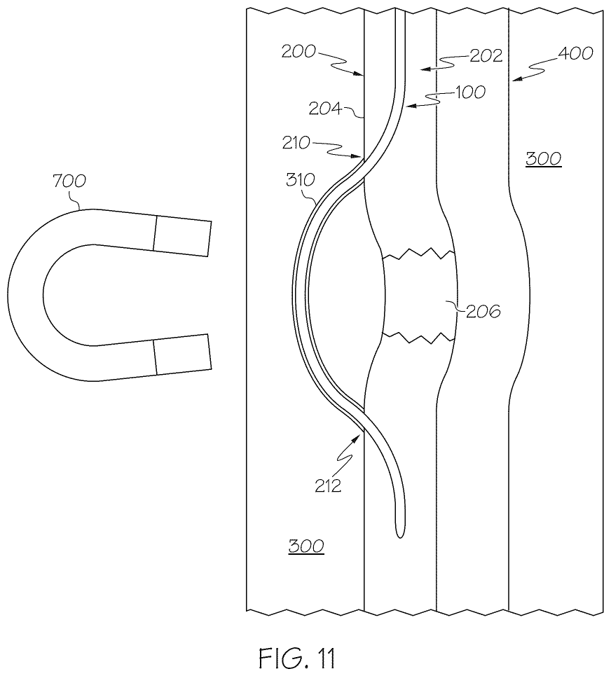

For instance, with reference to FIG. 11, a method of treating a chronic occlusion with the guidewires 100, 150 will be discussed. A first blood vessel 200 includes an occlusion 206 within a lumen 202 of the first blood vessel 200. The first blood vessel 200 may be an artery or a vein. A second blood vessel 400 may be a concomitant vessel of the first blood vessel 200. The second blood vessel 400 may be an artery or a vein. Current bypass methods to bypass the occlusion 206 in the first blood vessel 200 generally include hijacking the second blood vessel 400. That is, incisions, ablations, or the like may be made in the first blood vessel 200 and the second blood vessel 400. A stent or graft may then be passed between the first blood vessel 200 and the second blood vessel 400 at a first point, ran through the second blood vessel 400 to bypass the occlusion 206, and passed back between the second blood vessel 400 and the first blood vessel 200. The stent or graft redirects blood flow in the first blood vessel 200 around the occlusion 206. However, by running through a length of the second blood vessel 400, the second blood vessel 400 is harmed and the stent or graft may negatively impact natural blood flow within the second blood vessel 400.

Methods incorporating the guidewires 100, 150 herein address one or more of the shortcomings of current procedures. While the guidewire 100 will be specifically referenced in detail when discussing the methods disclosed herein, it should be appreciated that any embodiment of guidewire 100, 150 discussed with reference to FIGS. 1-10 may be utilized in the disclosed methods. Referring to FIGS. 1-6 and 11, the guidewire 100 is positioned within the first blood vessel 200 including the occlusion 206. The guidewire 100 may be inserted into the first blood vessel 200 upstream of the occlusion 206 and directed through the lumen 202 of first blood vessel 200 toward the occlusion 206. At a first point 210 upstream of the occlusion 206, the guidewire 100 may be directed into tissue 300 surrounding the first blood vessel 200. Particularly, the guidewire 100 may be tunneled through a wall 204 of the first blood vessel 200 and into the tissue 300 surrounding the first blood vessel 200 at the first point 210. The guidewire may be tunneled through the tissue 300 a distance such that the occlusion 206 is bypassed by the tip 103 of the guidewire 100. The guidewire 100 may then be directed into the first blood vessel 200 at a second point 212. Particularly, the guidewire 100 may be tunneled through the wall 204 of the first blood vessel 200 and into the lumen 202 of the first blood vessel 200 at the second point 212. The guidewire 100 may, therefore, form a passageway 310 through the tissue 300 surrounding the first blood vessel 200 from the first point 210 to the second point 212. The occlusion 206 may be positioned between the first point 210 and the second point 212. It should be appreciated that the guidewire 100 may traverse any desirable distance through the tissue 300 between the first point 210 and the second point 212. For instance, the length of the passageway 310 formed by the guidewire 100 through the tissue 300 may be greater than or equal to 1 mm, greater than or equal to 5 mm, greater than or equal to 7 mm, greater than or equal to 10 mm, greater than or equal to 15 mm, greater than or equal to 20 mm, greater than or equal to 30 mm, greater than or equal to 40 mm, greater than or equal to 50 mm, greater than or equal to 60 mm, greater than or equal to 75 mm, or greater than or equal to 100 mm.

In embodiments the tissue 300 may be any non-vascular tissue. The tissue 300 may be any fascia of tissue surrounding the first blood vessel. The tissue 300 may be a pedicle bundle, which is generally a fascia of connective tissue that surrounds and maintains concomitant arteries and veins, as well as nerve bundles. The guidewire 100 may be particularly configured to tunnel and form the passageway 310 through the tissue 300 by means of the RF energy delivered to the tip 103 of the guidewire 100. Particularly, the RF energy delivered to the tip 103 of the guidewire 100 allows the guidewire 100 to ablate the tissue 300 while puncturing and/or tunneling through the tissue 300. Moreover, the increased steerability of the guidewire 100 offers a user sufficient control to accurately tunnel and form the passageway 310 through the tissue 300, where the guidewire 100 may otherwise be more difficult to precisely maneuver than if the guidewire 100 were within a lumen of a blood vessel (e.g. the lumen 202 of the first blood vessel 200). Particularly, a user may steer the guidewire 100 with the first directional fiber 112 and/or the second directional fiber 114, discussed above. In embodiments, a user may steer the guidewire 100 by means of an external magnet 700. The external magnet 700 may be positioned outside the body of the patient having the occlusion 206. The external magnet 700 may particularly act on the core 106 of the guidewire 100. A user may manipulate the external magnet 700 to steer the guidewire 100 in a precise direction. In embodiments, a user may steer the guidewire 100 by means of both the directional fibers 112, 114 and/or the external magnet 700.

Referring now to FIGS. 1-6 and 11-13, a user may pass a catheter 710 over the guidewire 100. A user may pass the catheter 710 over the guidewire 100 and through at least a portion of the passageway 310. Mounted to the catheter 710 may be an expansion device 712 configured to radially expand around the catheter 710. The expansion device 712 may be a balloon configured to radially expand about the catheter 710. In some embodiments, the expansion device 712 may be integrated into the catheter 710 such as in a balloon catheter. A stent graft 500 may be mounted to the expansion device 712 such that when the expansion device 712 radially expands about the catheter 710, the stent graft 500 also radially expands about the catheter 710. For instance, and with particular reference to FIG. 12, which depicts a sectional view of the stent graft 500 for case of illustration, the expansion device 712 may radially expand outwardly from the catheter 710 to simultaneously radially expand the stent graft 500 in the direction of the walls of the passageway 310. While embodiments including the expansion device 712 have been discussed herein, it should be appreciated that the stent graft 500 may be deployable into a radially expanded state by any other desirable method or configuration. For example, in embodiments, the stent graft 500 may be naturally biased to an expanded state, and a sleeve associated with the catheter 710 may maintain the stent graft 500 against the catheter 710 until the stent graft 500 is desirably positioned for deployment, at which point, the sleeve may be removed and the stent graft 500 may radially expand in the direction of the walls of the passageway 310.

In addition to the catheter 710, the stent graft 500 is also passed over the guidewire 100 and through at least a portion of the passageway 310. The length of the stent graft 500, and the positioning of the stent graft 500 when the stent graft 500 is deployed, that is the positioning of the catheter 710 and stent graft 500 when the stent graft 500 is transitioned to a radially expanded state, may be such that that the stent graft 500 extends through the passageway 310 from the first point 210 to the second point 212. The stent graft 500 may enhance the stability of the passageway 310. That is, the stent graft 500 may prevent the narrowing or collapse of the passageway 310. Moreover, the stent graft 500, along with the expansion device 712 may increase the diameter of the passageway 310 formed by the guidewire 100, thereby promoting the flow of blood from the lumen 202 of the first blood vessel 200 into the passageway 310. The stent graft 500 may radially expand such that the stent graft 500 occupies the full volume of the passageway 310. The stent graft 500 provides an un-obstructed path for blood flow from the first point 210 to the second point 212. Particularly, blood may flow through the lumen 202 of first blood vessel 200 to the first point 210 upstream of the occlusion 206, through the stent graft 500 in the passageway 310 to the second point 212 downstream of the occlusion 206, and back into the lumen 202 of the first blood vessel 200. Therefore, the guidewire 100 and methods of using the same described herein provide means for bypassing the occlusion 206 that do not involve hijacking or otherwise disturbing the second blood vessel 400.

Embodiments have been discussed herein where the guidewire 100 is first inserted into the lumen 202 of the first blood vessel 200 such that the guidewire 100 tunnels the passageway 310 from the first point 210 to the second point 212. This is a non-limiting example, however. For instance, the guidewire 100 may be inserted into the lumen 202 of the first blood vessel 200 downstream of the occlusion 206 and directed through the lumen 202 of the first blood vessel 200 toward the occlusion 206. At the second point 212 downstream of the occlusion 206, the guidewire 100 may be directed into the tissue 300 surrounding the first blood vessel 200. Particularly, the guidewire 100 may be tunneled through a wall 204 of the first blood vessel 200 and into the tissue 300 surrounding the first blood vessel 200 at the second point 212. The guidewire 100 may be tunneled through the tissue 300 a distance such that the occlusion 206 is bypassed by the tip 103 of the guidewire 100. The guidewire 100 may then be directed into the first blood vessel 200 at the first point 210. Particularly, the guidewire 100 may be tunneled through the wall 204 of the first blood vessel 200 and into the lumen 202 of the first blood vessel 200 at the first point 210. The guidewire 100 may, therefore, form the passageway 310 through the tissue 300 surrounding the first blood vessel 200 from the second point 212 to the first point 210. The catheter 710 and stent graft 500 may then be similarly passed over the guidewire 100 as discussed above, but from a point downstream of the occlusion 206.

In additional methods, the guidewires 100, 150 herein, being configured to tunnel through particularly dense tissue, may also enable the formation of artificial connections between blood vessels spaced apart from each other. As one example, a fistula is generally a passageway formed between two internal organs. Forming a fistula between two blood vessels can have one or more beneficial functions, such as providing access to the vasculature for hemodialysis patients. Specifically, forming a fistula between an artery and a vein allows blood to flow quickly between the vessels while bypassing the capillaries. Less invasive methods of forming fistulas may include utilization of catheters in adjacent blood vessels that a fistula is to be formed between. One challenging aspect of forming a fistula between blood vessels, however, is properly aligning and coapting catheters in adjacent blood vessels prior to fistula formation. This may become increasingly challenging based on a distance between the blood vessels, or an amount of non-vascular tissue separating the blood vessels. That is, depending on the distance of non-vascular tissue separating the blood vessels, traditional catheter alignment mechanisms may be unable to properly align and coapt the catheters. Moreover, traditional vessel modification elements (e.g., tissue ablation electrodes, cutting mechanism, and/or the like) may be unable to form a connection between the target vessels through the non-vascular tissue separating them.

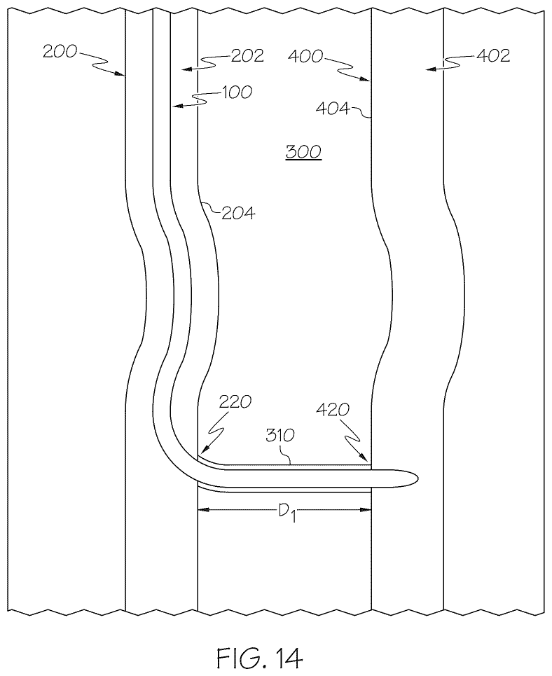

Methods incorporating the guidewires 100, 150 herein address one or more of the shortcomings of current procedures. While the guidewire 100 will be specifically referenced in detail when discussing the methods disclosed herein, it should be appreciated that any embodiment of guidewire 100, 150 discussed with reference to FIGS. 1-10 may be utilized in the disclosed methods. For instance, with reference to FIG. 14, a method of bridging a gap between vessels with the guidewires 100, 150 will be discussed. The first blood vessel 200 may be an artery or a vein. The second blood vessel 400 may an artery or a vein. The first blood vessel 200 and the second blood vessel 400 may be located in the anatomical snuff box. The first blood vessel 200 may be one of the cephalic vein or radial artery, and the second blood vessel 400 may be the other of the cephalic vein or radial artery. The wall 204 of the first blood vessel 200 and a wall 404 of the second blood vessel 400 may be separated by a distance D₁. The distance D₁ may be occupied by the tissue 300 separating the first blood vessel 200 and the second blood vessel 400. The tissue 300 separating the first blood vessel 200 and the second blood vessel 400 by the distance D1 may be non-vascular tissue. The tissue 300 may be any fascia of tissue separating the first blood vessel 200 and the second blood vessel 400. The tissue 300 may be a pedicle bundle.

With reference to FIGS. 1-6 and 14, the guidewire 100 may be positioned within the lumen 202 of the first blood vessel 200. The guidewire 100 may be directed through the first blood vessel 200 to a first point 220. At the first point 220, the guidewire 100 may be directed into tissue 300 separating the first blood vessel 200 and the second blood vessel 400. Particularly, the guidewire 100 may be tunneled through the wall 204 of the first blood vessel 200 and into the tissue 300 at the first point 220. The guidewire 100 may be tunneled through the tissue 300 the distance D₁ to a second point 420 of the second blood vessel 400. The guidewire 100 may further be directed through the wall 404 of the second blood vessel 400 at the second point 420. Particularly, the guidewire 100 may be tunneled through the wall 404 of the second blood vessel 400 and into a lumen 402 of the second blood vessel 400 at the second point 420. The guidewire 100 may therefore form a passageway 310 from the first blood vessel 200 to the second blood vessel 400 through the tissue 300 separating the first blood vessel 200 and the second blood vessel 400.

The first point 220 of the first blood vessel 200 and the second point 420 of the second blood vessel 400 are separated by the distance D₁. The distance Dᵢ may be less than or equal to 2 mm. The distance Dᵢ may be greater than or equal to 2 mm. The distance D₁ may be greater than or equal to 3 mm. The distance Dᵢ may be greater than or equal to 5 mm. It is noted that larger or smaller distances are contemplated and possible.

As discussed previously, the guidewire 100 may be particularly configured to tunnel and form the passageway 310 through the tissue 300 by means of the RF energy delivered to the tip 103 of the guidewire 100. Moreover, the increased steerability of the guidewire 100 offers a user sufficient control to accurately tunnel and form the passageway 310 through the tissue 300, where the guidewire 100 may otherwise be more difficult to precisely maneuver than if the guidewire 100 were within a lumen of a blood vessel (e.g. the lumen 202 of the first blood vessel 200). Particularly, a user may steer the guidewire 100 with the first directional fiber 112 and/or the second directional fiber 114, discussed above. In embodiments, a user may steer the guidewire 100 by means of the external magnet 700 (FIG. 11). In embodiments, a user may steer the guidewire 100 by means of the directional fibers 112, 114 and/or the external magnet 700 (FIG. 11).

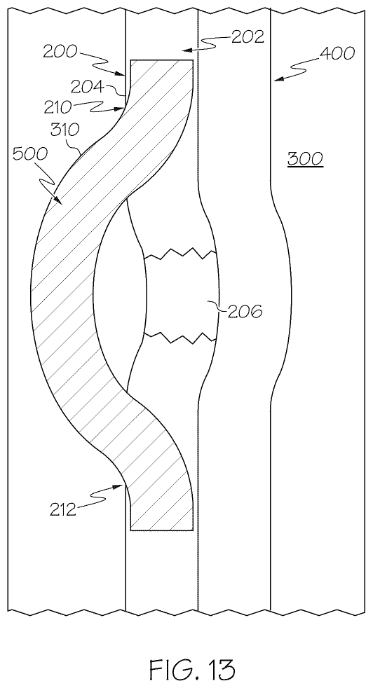

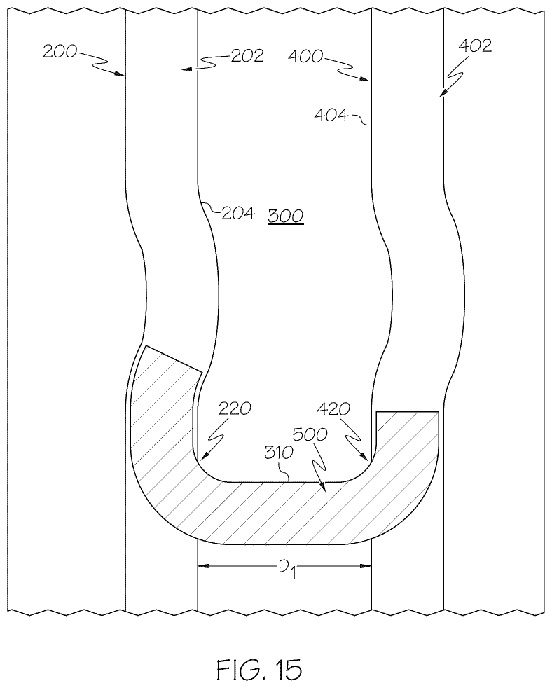

As discussed in FIGS. 12 and 13, a user may pass a catheter 710 (FIG. 12) over the guidewire 100 and through at least a portion of the passageway 310. A stent graft 500 may be mounted on and deployable from the catheter 710 (FIG. 12), as previously discussed. In addition to the catheter 710 (FIG. 12), the stent graft 500 is also passed over the guidewire 100 and through at least a portion of the passageway 310. With reference to FIG. 15, the length of the stent graft 500, and the positioning of the stent graft 500 when the stent graft 500 is deployed, that is the positioning of the catheter 710 (FIG. 12) and stent graft 500 when the stent graft 500 is transitioned to a radially expanded state, may be such that that the stent graft 500 extends through the passageway 310 from the first point 220 to the second point 420. The stent graft 500 may enhance the stability of the passageway 310. That is, the stent graft 500 may prevent the narrowing or collapse of the passageway 310. Moreover, the stent graft 500, along with the expansion device 712 may increase the diameter of the passageway 310 formed by the guidewire 100. The stent graft 500 may radially expand such that the stent graft 500 occupies the full volume of the passageway 310. The stent graft 500 provides an un-obstructed path for blood flow from the first point 220 to the second point 420. Particularly, blood may flow through the lumen 202 of first blood vessel 200 to the first point 220, through the stent graft 500 in the passageway 310 to the second point 420, and into the lumen 402 of the second blood vessel 400. It should be appreciated, that blood may instead flow from the second point 420 through the stent graft 500 in the passageway 310 to the first point 220.

With reference now to FIGS. 1-6, 14, 16, and 17, a fistula may be formed between the first point 220 of the first blood vessel 200 and the second point 420 of the second blood vessel 400 following formation of the passageway 310. As discussed previously, the tissue 300 separating the first blood vessel 200 and the second blood vessel 400 by the distance D₁ may limit traditional fistula-forming devices in their ability to properly align and coapt in the first blood vessel 200 and the second blood vessel 400. Moreover, the tissue 300 separating the first blood vessel 200 and the second blood vessel 400 by the distance D₁ may limit traditional fistula-forming devices in their ability to modify the first blood vessel 200 and/or the second blood vessel 400. By eliminating the tissue 300 in the passageway 310 with the guidewire 100, however, a fistula may more easily be formed between the first point 220 of the first blood vessel 200 and the second point 420 of the second blood vessel 400.

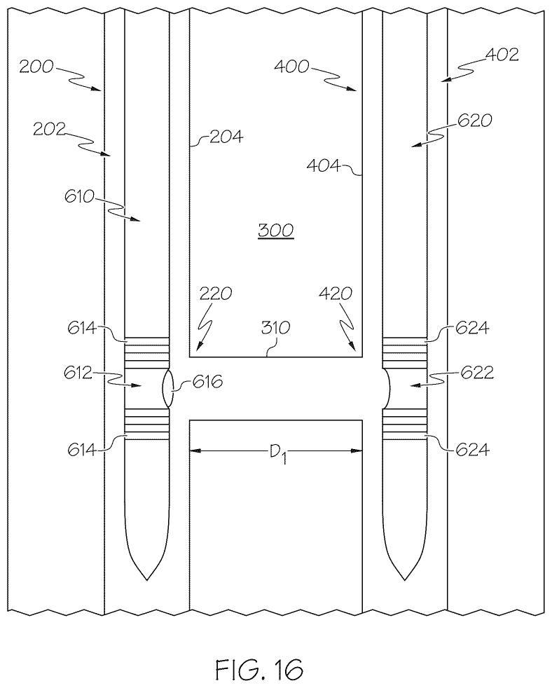

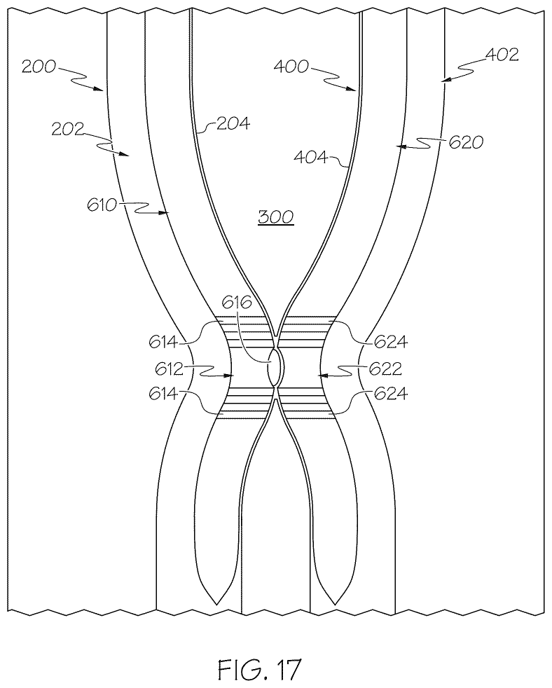

Merely as an example, and as shown in FIG. 16, a first catheter 610 may be advanced through the first blood vessel 200 and a second catheter 620 may be advanced through the second blood vessel 400. The first catheter includes a working site 612, configured to modify a blood vessel. For instance, a modification device 616, such as an electrode, ultrasonic cutting element, laser, knife, etc. may project from the working site 612 to modify a vessel. The second catheter 620 also includes a working site 622, which may be configured to receive the modification device 616 of the first catheter 610. The first catheter 610 may further include one or more magnets 614 and the second catheter 620 may include one or more magnets 624. The magnets 614, 624 may promote proper alignment between the working sites 612, 622 for formation of a fistula therebetween. The magnets 614, 624 may also promote strong coaptation between the working sites 612, 622. When in strong coaptation, as depicted in FIG. 17, the working sites 612, 622 are in close approximation such that the modification device 616 of the first catheter 610 may enter the recess of the working site 622 of the second catheter 620.

Alignment and coaptation of the first and second catheters 610, 620, and therefore the ability to accurately form a fistula between the first blood vessel 200 and the second blood vessel 400 at the first point 220 and the second point 420 may be enabled by the passageway 310 formed by the guidewire 100. That is, by eliminating the tissue 300 in the passageway 310, forces from the tissue 300 counteracting the magnetic attraction forces between the magnets 614, 624 may be reduced or eliminated. Therefore, the first and second catheters 610, 620 may be more accurately aligned and coapted, as shown in FIG. 17. Moreover, by eliminating the tissue 300 in the passageway 310, the amount and density of tissue that the modification device 616 must modify to form a fistula between the first blood vessel 200 and the second blood vessel 400 at the first point 220 and the second point 420 is reduced.

While embodiments of forming fistulas with catheters including magnets have been discussed in detail for illustrative purposes, it should be appreciated that this is merely an example. Any other fistula-forming catheters or devices may be used to form the fistula between the first point 220 and the second point 420, the operation of any of which may be strengthened by eliminating the tissue 300 in the passageway 310.

In any of the methods discussed above, the tip 103 of the guidewire 100 may be raised to different energy levels throughout the process of forming the passageway 310. For instance, the tip 103 of the guidewire 100 may be raised to a first energy level to puncture through a wall of the first blood vessel 200 into the tissue 300. The tip 103 of the guidewire 100 may then be operated at a second energy level to tunnel through the tissue 300. The second energy level may be less than the first energy level. Any desired second energy level may be selected based on the type of tissue 300 being tunneled through, the distance being tunneled through the tissue 300, and/or the density of the tissue 300 being tunneled through.

While embodiments have been discussed herein where the disclosed guidewires may be used in bypass and fistula forming procedures, it should be appreciated that these are merely non-limiting examples. That is, the guidewires discloses herein may be used in any desirable medical procedure, such as arterial bypass, venous bypass, arteriovenous fistula formation, hemodialysis, arterialization, and anastomosis. Moreover, it should be appreciated that the guidewires disclosed herein may be used to tunnel between any two points in a body. For instance, while embodiments discussed in detail include tunneling through non-vascular tissue from a first point of a first blood vessel to either a second point of the first blood vessel or a first point of a second blood vessel, it should be appreciated that these are non-limiting examples. That is, the guidewires discussed herein may be used to tunnel between any two points, that may or may not be of a blood vessel, as needed.

Embodiments can be described with reference to the following numerical clause:
1. A guidewire, comprising: a body; and a tip, wherein: the tip is configured to be energized by an RF generator connectable to the body; and the guidewire is configured to: tunnel through an internal lumen of a blood vessel; tunnel through a wall of the blood vessel; and tunnel through non-vascular tissue outside of the blood vessel.
2. The guidewire of clause 1, wherein the guidewire is configured to tunnel through a pedicle bundle outside of the blood vessel.
3. The guidewire of any preceding clause, wherein the guidewire is configured to tunnel through at least a 2 mm distance of the non-vascular tissue.
4. The guidewire of any preceding clause, wherein the guidewire is configured to tunnel through at least a 3 mm distance of the non-vascular tissue.
5. The guidewire of any preceding clause, wherein the guidewire is configured to tunnel through at least a 5 mm distance of the non-vascular tissue.
6. The guidewire of any preceding clause, wherein the guidewire comprises a
refractory metal.
7. The guidewire of any preceding clause, wherein the guidewire comprises tungsten.
8. The guidewire of any preceding clause, wherein the guidewire comprises a ferrous metal.
9. The guidewire of any preceding clause, wherein the guidewire comprises a ferrous metal core disposed within a shell of tungsten.
10. The guidewire of any preceding clause, wherein the guidewire is configured to be directed by a magnet.
11. The guidewire of any preceding clause, wherein the guidewire comprises an internal lumen, and a directional fiber disposed at least partly within the internal lumen.
12. The guidewire of any preceding clause, wherein the guidewire comprises a directional fiber coupled to an exterior of the guidewire body.
13. The guidewire of any preceding clause, wherein the guidewire is configured to: tunnel through the wall of the blood vessel at a first point; tunnel through the non-vascular tissue outside of the blood vessel; and tunnel through the wall of the blood vessel at a second point to form a passageway through the non-vascular tissue from the first point to the second point.
14. The guidewire of any preceding clause, wherein the guidewire is configured to: tunnel through the wall of the blood vessel at a first point; tunnel through the non-vascular tissue outside of the blood vessel; and tunnel through a wall of a second blood vessel at a second point to form a passageway from the first point to the second point through the non-vascular tissue.
15. A method of bypassing an occlusion, comprising: inserting a guidewire into a vessel; directing the guidewire into tissue surrounding the vessel at a first point; tunneling through the tissue surrounding the vessel with the guidewire; and directing the guidewire into the vessel at a second point to form a passageway through the tissue surrounding the vessel from the first point to the second point, wherein the guidewire is RF energized.
16. The method of clause 15, wherein the occlusion is positioned between the first point and the second point in the vessel.
17. The method of any preceding clause, wherein the tissue surrounding the vessel is a pedicle bundle.
18. The method of any preceding clause, wherein the vessel is an artery.
19. The method of any preceding clause, further comprising passing a catheter over the guidewire and through at least a portion of the passageway.
20. The method of any preceding clause, further comprising passing a stent graft over the guidewire and through at least a portion of the passageway.
21. The method of any preceding clause, wherein the stent graft forms a path for blood flow from the first point to the second point.
22. The method of any preceding clause, wherein the guidewire comprises a refractory metal.
23. The method of any preceding clause, wherein the guidewire comprises tungsten.
24. The method of any preceding clause, wherein the guidewire comprises a ferrous metal.
25. The method of any preceding clause, wherein the guidewire comprises a ferrous metal core disposed within a shell of tungsten.
26. The method of any preceding clause, wherein the guidewire is directed by a magnet.
27. The method of any preceding clause, wherein the guidewire comprises an internal lumen, and a directional fiber disposed at least partly within the lumen.
28. The method of any preceding clause, wherein the guidewire comprises a directional fiber coupled to an exterior of the guidewire.
29. A method of bridging a gap between a first vessel and a second vessel, comprising: inserting a guidewire into the first vessel; directing the guidewire out of the first vessel at a first point; tunneling through tissue separating the first vessel and the second vessel with the guidewire; and directing the guidewire into the second vessel at a second point to form a passageway from the first vessel to the second vessel through the tissue separating the first vessel and the second vessel, wherein the guidewire is RF energized.
30. The method of clause 29, wherein the first point of the first vessel and the second point of the second vessel are separated by at least 2 mm.
31. The method of any preceding clause, wherein the first point of the first vessel and the second point of the second vessel are separated by at least 3 mm.
32. The method of any preceding clause, wherein the first point of the first
vessel and the second point of the second vessel are separated by at least 5 mm.
33. The method of any preceding clause, further comprising forming a fistula between the first vessel and the second vessel between the first point and the second point.
34. The method of any preceding clause, further comprising passing a stent graft over the guidewire and through at least a portion of the passageway.
35. The method of any preceding clause, wherein the guidewire comprises a refractory metal.
36. The method of any preceding clause, wherein the guidewire comprises tungsten.
37. The method of any preceding clause, wherein the guidewire comprises a ferrous metal.
38. The method of any preceding clause, wherein the guidewire comprises a ferrous metal core disposed within a shell of tungsten.
39. The method of any preceding clause, wherein the guidewire is directed by a magnet.
40. The method of any preceding clause, wherein the guidewire comprises an internal lumen, and a directional fiber disposed at least partly within the lumen.
41. The method of any preceding clause, wherein the guidewire comprises a directional fiber coupled to an exterior of the guidewire.

It should now be understood that embodiments of the present disclosure are directed to RF energized guidewires. In particular, the guidewires described herein may include a body and an RF energized tip. The guidewires herein may be particularly configured to tunnel through an internal lumen of a blood vessel, tunnel through a wall of the blood vessel, and tunnel through non-vascular tissue outside of the blood vessel. The guidewires herein may tunnel through dense non-vascular tissue for large distances. Using the guidewires herein to tunnel through the greater distances or densities of tissue may enable bypass procedures where a stent graft is passed from a first point of an occluded blood vessel, through a passageway in surrounding tissue formed by the guidewire, and to a second point of the occluded blood vessel to bypass the occlusion. Using the guidewires herein to tunnel through the greater distances or densities of tissue may enable fistula-forming procedures where a fistula is formed between a first vessel and a second vessel separated by up to 5 mm of tissue. To increase the user control of the guidewires herein, the guidewires may be steered by an external magnet and/or directional fibers running through at least part of the length of the guidewires.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A guidewire, comprising:
a body; and
a tip, wherein:
the tip is configured to be energized by an RF generator connectable to the body; and
the guidewire is configured to:
tunnel through an internal lumen of a blood vessel;
tunnel through a wall of the blood vessel; and
tunnel through non-vascular tissue outside of the blood vessel.

2. The guidewire of claim 1, wherein the guidewire is configured to tunnel through a pedicle bundle outside of the blood vessel.

3. The guidewire of claim 1, wherein the guidewire is configured to tunnel through at least a 2 mm distance of the non-vascular tissue.

4. The guidewire of claim 1, wherein the guidewire is configured to tunnel through at least a 3 mm distance of the non-vascular tissue.

5. The guidewire of claim 1, wherein the guidewire is configured to tunnel through at least a 5 mm distance of the non-vascular tissue.

6. The guidewire of claim 1, wherein the guidewire comprises a refractory metal.

7. The guidewire of claim 1, wherein the guidewire comprises tungsten.

8. The guidewire of claim 1, wherein the guidewire comprises a ferrous metal.

9. The guidewire of claim 1, wherein the guidewire comprises a ferrous metal core disposed within a shell of tungsten.

10. The guidewire of claim 1, wherein the guidewire is configured to be directed by a magnet.

11. The guidewire of claim 1, wherein the guidewire comprises an internal lumen, and a directional fiber disposed at least partly within the internal lumen.

12. The guidewire of claim 1, wherein the guidewire comprises a directional fiber coupled to an exterior of the guidewire body.

13. The guidewire of claim 1, wherein the guidewire is configured to:
tunnel through the wall of the blood vessel at a first point;
tunnel through the non-vascular tissue outside of the blood vessel; and
tunnel through the wall of the blood vessel at a second point to form a passageway through the non-vascular tissue from the first point to the second point.

14. The guidewire of claim 1, wherein the guidewire is configured to:
tunnel through the wall of the blood vessel at a first point:
tunnel through the non-vascular tissue outside of the blood vessel; and
tunnel through a wall of a second blood vessel at a second point to form a passageway from the first point to the second point through the non-vascular tissue.

15. A method of bypassing an occlusion, comprising:
inserting a guidewire into a vessel;
directing the guidewire into tissue surrounding the vessel at a first point:
tunneling through the tissue surrounding the vessel with the guidewire; and
directing the guidewire into the vessel at a second point to form a passageway through the tissue surrounding the vessel from the first point to the second point, wherein the guidewire is RF energized.

16. The method of claim 15, wherein the occlusion is positioned between the first point and the second point in the vessel.

17. The method of claim 15, wherein the tissue surrounding the vessel is a pedicle bundle.

18. The method of claim 15, wherein the vessel is an artery.

19. The method of claim 15, further comprising passing a catheter over the guidewire and through at least a portion of the passageway.

20. The method of claim 15, further comprising passing a stent graft over the guidewire and through at least a portion of the passageway.

21.-41. (canceled)
